(19) United States
(12) Plant Patent Application Publication
(10) Pub. No.: US 2015/0351298 P1
(43) Pub. Date: Dec. 3, 2015

(54) Thuja plant named 'MonRig'

(50) ;

(71) Applicant: Rigoberto Pineda, Salem, OR (US)

(72) Inventor: Rigoberto Pineda, Salem, OR (US)

(21) Appl. No.: 14/120,543

(22) Filed: Jun. 2, 2014

(51) Int. Cl. A01H 7/00

(52) U.S. Cl. PLT/213

(57) ABSTRACT

A new and distinct Thuja cultivar named ‘MonRig’ is disclosed, characterized by distinctive Yellow-Green and Green foliage, which changes to chocolate brown in the Winter. Plants have distinctive tight, upright growth habit. The new cultivar is a Thuja, suitable for ornamental garden purposes.

### LATIN NAME OF THE GENUS AND SPECIES

Thuja compacta

### VARIETY DENOMINATION

‘MonRig’

### BACKGROUND OF THE INVENTION

The new cultivar is a product of chance discovery by the inventor. This new variety, hereinafter referred to as ‘MonRig’, was discovered as a naturally occurring, whole plant mutation by the inventor, Rigoberto Pineda. The interesting new mutation was discovered in a commercial nursery in Dayton, Oreg. among a population of unpatented Thuja compacta during June of 2010.

After identifying the new variety as a potentially interesting selection, the inventor first organized propagation of ‘MonRig’ by tissue culture at the same commercial laboratory during December of 2010. The inventor continued controlled testing and propagation, assessing stability of the unique characteristics of this variety. At least five generations have been reproduced and have shown that the unique features of this cultivar are stable and reproduced true to type.

### SUMMARY OF THE INVENTION

The cultivar ‘MonRig’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘MonRig’ These characteristics in combination distinguish ‘MonRIG’ as a new and distinct Thuja cultivar:

1. Unique color of foliage:
Youngest new foliage is yellow-green, changing to green. Foliage is chocolate brown in the Winter.

2. Distinctive tight, compact growth.

3. Distinctive upright branching.

### COMPARISON TO PARENT VARIETY

‘MonRig’ is similar in most horticultural characteristics to the parent variety Thuja compacta. Plants of the new cultivar ‘MonRig’ however, grow more slowly, and are more compact with more upright branching. Additionally, the parent variety does not have chocolate brown winter foliage.

### COMMERCIAL COMPARISON

To the breeder's best information, ‘MonRig’ can best be compared to the parent variety.

### BRIEF DESCRIPTION OF THE PHOTOGRAPHS

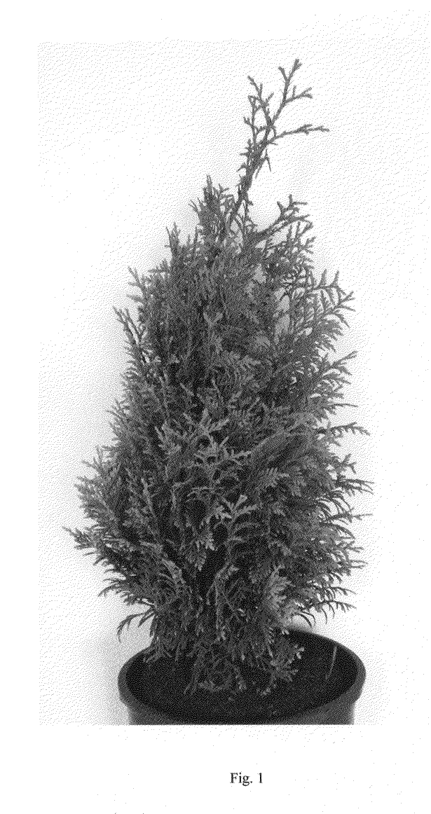

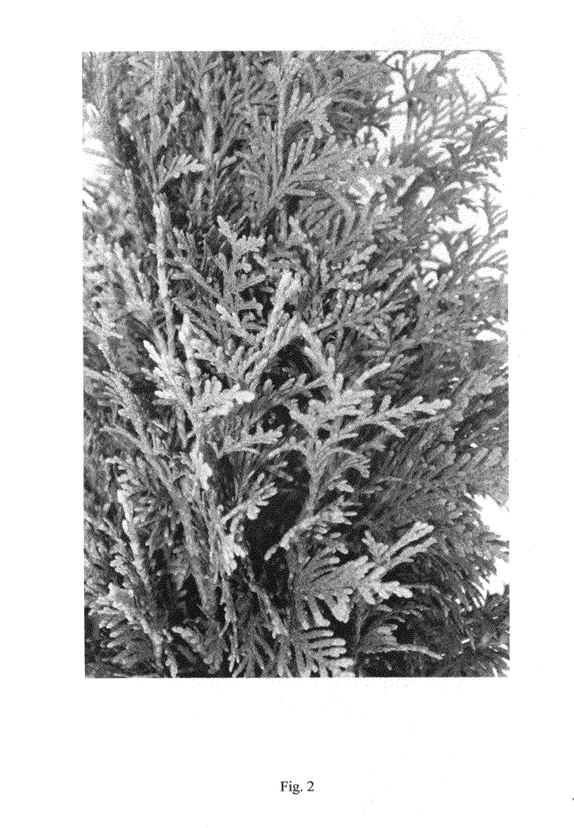

The accompanying photograph in FIG. 1 illustrates in full color a typical plant of ‘MonRig’ grown outdoors in Magnolia, Tex. The plant is approximately 2 years old, and is shown in a three gallon container. FIG. 2 shows a close up of typical foliage of the new variety. The photographs were taken using conventional techniques and although colors may appear different from actual colors clue to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘MonRig’ plants grown outdoors in Dayton, Oreg. Plants are approximately 2 years old, in a 3 gallon nursery container. Measurements and numerical values represent averages of typical plant types.
Botanical classification: Thuja compacta ‘MonRig’
Propagation:
   Time to rooting.—60 to 90 days at approximately 40-50° F.
   Root description.—Fibrous, very well branched. Colored near RHS Brown 200C.
Plant:
   Growth habit.—Tightly pyramidal to conical, uniform appearance.
   Height.—Approximately 152 cm. after 4 years.
   Plant spread.—Approximately 56 cm. after 4 years.
   Typical growth rate.—Moderate for species.
   Pinching required.—Yes Details of Pinching: Light shearing to maintain shape.
   Branching characteristics.—Branching arrangements. Alternate, main stem is upright with numerous (approximately 40) secondary branches. Lateral stems are held at an angle of 45 degree with flat sprays extending upward.
   Length of lateral branches.—Approximately 18 cm.
   Average number of lateral branches.—Approximately 5 main branches, approximately 40 secondary branches.
   Diameter of lateral branches.—Approximately 0.5 cm.
   Lateral branch shape in cross section.—Generally oval.
   Lateral branch strength.—Flexible.
   Lateral branch color.—Near RHS Greyed-Orange 177B.
   Lateral branch texture.—Scaly and soft to the touch. Scales colored near Greyed-Orange 177C and 177D.
   Internode length.—Approximately 4.5 cm on average of main stem approximately 3 cm on average approaching the tip.
   Lenticels present.—No.
Foliage:
   General description.—Tightly held, scale like leaves on planar branchlets.
   Leaf.—Arrangement: Opposite Type: Scale Shape of blade: Awl shaped. Average Length: Approximately 0.25 cm. Average Width: Approximately 0.15 cm. Apex: Acute. Base: Truncate. Attachment: Sessile Margin: Entire Texture of top surface: Glabrous Texture of bottom surface: Glabrous Leaf internode length: Leaves touching, overlapping as younger leaves emerge above older foliage. Color: Young foliage upper side: Near R.H.S. Yellow-Green 151 B. Young foliage under side: Near R.H.S. Yellow-Green 151B. Mature foliage upper side: Near R.H.S. Green 137A Mature foliage under side: Near R.H.S. Green 137A Venation: Linear, indistinguishable from leaf blade. Durability of foliage to stresses: Flexible and resistant to breakage. Fragrance: Strong, pleasant cedar foliage-like scent.
   Branchlets.—completely covered by leaves. Dissected into 2 or 3 sub-branchlets. Average Length: Approximately 7.5 cm. Average Width: Approximately 3.5 cm. Density of leaves: Approximately 10 leaves per cm.
Flower: Flowering not observed.
Other characteristics:
   Disease and pest resistance.—Neither resistance nor susceptibility observed.
   Drought tolerance.—No tolerance for drought.
   Temperature tolerance.—USDA Zones 4-8.

What is claimed is:

1. A new and distinct cultivar of Thuja plant named ‘MonRig’ as herein illustrated and described.
